(19) United States
(12) Patent Application Publication
(10) Pub. No.: US 2023/0106068 A1
(43) Pub. Date: Apr. 6, 2023

(54) AN EFFECTIVE ANTI-ACNE PERSONAL CARE COMPOSITION

(71) Applicant: Conopco, Inc., d/b/a UNILEVER, Englewood Cliffs, NJ (US)

(72) Inventors: Sayandip MUKHERJEE, Bangalore (IN); Ajay Singh NEGI, Bangalore (IN); Bharath PALANISAMY, Bangalore (IN); Sandip Bhanudas PATHAK, Bangalore (IN)

(73) Assignee: Conopco, Inc., d/b/a UNILEVER, Englewood Cliffs, NJ (US)

(21) Appl. No.: 17/801,393

(22) PCT Filed: Feb. 18, 2021
(86) PCT No.: PCT/EP2021/054002

§ 371 Date: Aug. 22, 2022

(30) Foreign Application Priority Data
Mar. 2, 2020 (EP) 20160349.5

(51) Int. Cl. A61K 8/66; A61K 8/99; A61P 17/10; A61P 31/04; A61Q 19/00; A61K 8/73; A61K 8/20

(52) U.S. Cl. CPC A61K 8/66; A61K 8/99; C12Y 302/01017; A61P 17/10; A61P 31/04; A61Q 19/00; A61K 8/731; A61K 8/20; A61K 2800/30; A61K 2800/5422

(57) ABSTRACT

The present invention relates to a method and a composition to prevent or treat acne by selective inhibition of P. acnes bacteria. This is delivered using a composition comprising P. acnes phage-derived endolysins where the actives are maintained at conditions like use of non-ionic polymers and specific pH and electrolytes range that ensure maximal inhibition efficacy.

### FIELD OF THE INVENTION

The present invention relates to a method and a composition to prevent or treat acne by selective inhibition of P. acnes bacteria. This is delivered using a composition comprising P. acnes phage-derived endolysins where the actives are maintained at conditions that ensure maximal inhibition efficacy.

### BACKGROUND OF THE INVENTION

Acne, also known as Acne vulgaris, is a common skin condition that affects nearly all adolescents and adults at some point in their lives. It has a complex etiology, involving abnormal keratinization, excess sebum production, androgen function, bacterial growth, and immune hypersensitivity. Although one or more of the above processes is correlated with acne, the one triggering factor and the exact sequence of events leading to the formation of acne lesions has not been fully understood. Other factors which have been linked to acne are presence of free radicals with subsequent oxidative stress leading to cellular damage. It has been observed that acne usually occurs in areas rich in sebaceous glands like the face, neck and back.

A bacteria Propionibacterium acnes (P. acnes) has also been implicated in occurrence of acne. It is one of the important and dominant bacteria residing on the human facial skin surface. P. acnes is an aerotolerant anaerobe, slow-growing, rod shaped Gram-positive bacteria. It resides in the sebaceous glands. P. acnes uses sebum and by-product from surrounding skin tissue as sources of energy and nutrients. This results in some fatty acid release which can irritate the follicle wall and induce inflammation, leading to acne or acne vulgaris. Acne vulgaris is a chronic, inflammatory disorder of the pilosebaceous gland. It affects almost all adolescents at some point of their lives with 15-20% suffering from moderate to severe forms of acne.

Acne has been treated in many ways. Most treatments take several weeks to months before a noticeable change is seen. Benzoyl peroxide which has an antibacterial effect has been used for mild cases of comedones. In very severe cases of acne, antibiotics like tetracycline, erythromycin and clindamycin have been used. Antibiotics are believed to work by several mechanisms, the most important being the decrease in the number of bacteria in and around the follicle. They are also thought to reduce the irritating chemicals produced by the white blood cells in the sebum, thereby reducing the inflammatory response. However, the drawback of antibiotics and other sort of general antimicrobial treatment is that they are broad-spectrum and help in killing or inhibiting most of the bacteria on skin.

Most of the treatments, as summarized above, involve use of synthetic chemicals which many people believe to be harsh on the human body. More and more people prefer use of materials which are “natural” e.g. actives based on herbal origin. Further, these days, the actives based on microbiological or biotechnological origin is also coming to be considered as more “natural” then purely synthetic approaches used so far.

Skin, human body's largest organ is colonized by diverse microorganisms. Most of the microorganisms are considered either beneficial or opportunistic potential pathogens. Interactions between skin microorganisms and the human host can be classified into three clusters: 1. Commensalism (one benefits, and no harm occurs to the other), 2. Mutualism (both find benefit), 3. Parasitism (one organisms benefit and other is harmed). Microbes which are not often associated with disease, are mainly known as commensals. Increasing evidence shows that commensal organisms on skin help the immune system to fight against pathogens and maintain homeostasis of the microbiome. One example is Staphylococcus epidermidis (a Gram-positive coagulase negative staphylococci) which plays an important role in acne inhibition. Staphylococcus epidermidis mediates fermentation of glycerol which inhibit overgrowth of P. acnes, leading to acne inhibition. Thus, the approach to use broad spectrum antibiotic action which kills or inhibits all the microorganisms on the skin is finding lesser and lesser acceptance among health care practitioners. Increasingly, the approach to selective kill or inhibit microorganism on skin, is finding favour among health care practitioners.

Thus, there is a need for finding novel methods of treating problems such as acne in selective fashion. The present inventors have worked towards providing such selective methods in solving the problem of acne. They have filed a patent application published as WO2019/2380409 which discloses an isolate (extracellular factor) of Staphylococcus capitis ureolyticus (SCU), a minor commensal contributor of the skin flora, that was found to selectively inhibit the growth of P. acnes. In that invention, the efficacy was further improved by inclusion of P. acnes phage-derived endolysins. They then thereafter were looking for ways in which the efficacy of such compositions could be improved. After extensive trails they hit upon a selective cosmetically acceptable base which comprises non-ionic polymers which could retain and ensure maximal efficacy of the P. acnes phage derived endolysins. Such a composition was further found to work best in a specific pH range and at certain low concentrations of electrolytes.

It is thus an object of the present invention to provide for selective kill or inhibition of P. acnes to treat acne.

It is another object of the present invention to provide solution to treating acnes through more natural or nature based actives.

### SUMMARY OF THE INVENTION

According to the first aspect of the present invention there is provided an effective anti-acne personal care composition comprising
(i) P. acnes phage-derived endolysins or nucleic acid molecules encoding the same; and
(ii) A topically acceptable carrier comprising a non-ionic polymer.

According to another aspect of the present invention there is provided a method of controlling or eradicating P. acnes from skin comprising the step of applying a composition of the first aspect on to the desired skin surface.

### DETAILED DESCRIPTION OF THE INVENTION

These and other aspects, features and advantages will become apparent to those of ordinary skill in the art from a reading of the following detailed description and the appended claims. For the avoidance of doubt, any feature of one aspect of the present invention may be utilized in any other aspect of the invention. The word “comprising” is intended to mean “including” but not necessarily “consisting of” or “composed of.” In other words, the listed steps or options need not be exhaustive. It is noted that the examples given in the description below are intended to clarify the invention and are not intended to limit the invention to those examples per se. Similarly, all percentages are weight/weight percentages unless otherwise indicated.

Except in the operating and comparative examples, or where otherwise explicitly indicated, all numbers in this description indicating amounts of material or conditions of reaction, physical properties of materials and/or use are to be understood as modified by the word “about”. Unless specified otherwise, numerical ranges expressed in the format “from x to y” are understood to include x and y. When for a specific feature multiple preferred ranges are described in the format “from x to y”, it is understood that all ranges combining the different endpoints are also contemplated.

The composition as per this invention could be in the form of a leave-on or wash-off format meant for cleaning or disinfecting topical areas e.g. skin and/or hair of mammals, especially humans. Such a composition includes any product applied to a human body for also improving appearance, cleansing, odor control or general aesthetics. The composition of the present invention may be in the form of a liquid, lotion, cream, foam or gel, or toner, or applied with an implement or via a face mask, pad or patch. “Skin” as used herein is meant to include skin on the face and body (e.g., neck, chest, back, arms, underarms, hands, legs, buttocks and scalp). It is especially useful for treatment of acne on the face or any other part of the body where acne forms.

The present invention relates to an antimicrobial composition for selectively inhibiting growth of P. acnes bacteria comprising P. acnes phage-derived endolysins or nucleic acid molecules encoding the same; and a topically acceptable carrier comprising a non-ionic polymer. An especially preferred composition comprises P. acnes phage-derived endolysins.

By P. acnes is meant Propionibacterium acnes which is also known as Cutibacterium acnes.

The composition as per the present invention comprises P. acnes phage-derived endolysins or nucleic acid molecules encoding the same. Endolysins as per this invention are bacteriophage-derived enzymes. A group of phage-derived enzymes are peptidoglycan (PG) hydrolases known as endolysins. Endolysins are novel muralytic hydrolases encoded by double stranded DNA phages which degrade the PG layer of the bacterial cell wall thereby allowing the progeny phages to escape in the late phase of the infection cycle. Purified endolysins when exposed to PG externally can cause “lysis from outside”. Based on their antimicrobial properties (extraordinary substrate specificity and high activity when added externally) endolysins from phages infecting Gram-positive pathogens has been the motivation and hypothesis for the present inventors to ensure that the effect is exhibited to the best extent possible when formulated in a composition that is preferred by the consumer.

Thus, it is useful that the endolysin that is included is a recombinant form of P. acnes phage endolysin. This endolysis is preferably cloned from endolysin gene sequence (Gene ID: NC_018851; 855 nucleotide base pair long, 284 amino acids, protein ID: 97935.1) from Propionibacterium phage 29399B_C (GenBank: JX262225.1) which is codon optimized for expression in E. coli and cloned into commercially available pET303/CT-His expression vector.

The nucleotide sequence of endolysin which is especially useful is as per the sequence ID SEQ ID1 which is listed below:

ATGCGTTATATTCCGGCAGCACATCATTCAGCAGGTAGCAATCATCCGG
TTAATCGTGTTGTTATTCATGCAACCTGTCCGGATGTTGGTTTTCCGAG
CGCAAGCCGTAAAGGTCGTGCAGTTAGCACCGCAAACTATTTTGCAAGC
CCGAGCAGCGGTGGTAGCGCACATTATGTTTGTGATATTGGTGAAACCG
TTCAGTGTCTGAGCGAAGGCACCATTGGTTGGCATGCACCGCCTAATCC
GCATAGCCTGGGTATTGAAATTTGTGCAGATGGTGGTAGCCATGCAAGC
TTTCGTGTTCCGGGTCATGCATATACCCGTGAACAGTGGCTGGATCCGC
GTGTTTGGCCTGCAGTTGAAAAAGCAGCAATTCTGTGTCGTCGTCTGTG
CGATAAATACAATGTTCCGAAACGTAAACTGAGCGCAGCAGATCTGAAA
GCAGGTCGTCGTGGTGTTTGTGGTCATGTTGATGTTACCGATGCATGGC
ATCAGAGCGATCATGATGATCCGGGTCCGTGGTTTCCGTGGGATCGTTT
TATGGCAGTTGTTAATGGTCATAACGAAAGCGGTGAACTGACCGTTGCA
GATGTTAAAGCACTGCATGATCAGATTAAACAGCTGAGCGCACAGCTGG
CAGGTAGCGTTAATAAACTGCATCATGATGTTGGTGTTGTTCAGGTTCA
GAATGGTGATCTGGGTAAACGTGTTGATGCACTGAGCTGGGTTAAAAAT
CCGGTTACCGGTAAACTGTGGCGTACCAAAGATGCACTGTGGTCAGTTT
GGTATTATGTTCTGGAATGTCGTAGCCGTATTGATCGTCTGGAAAGCGC
AGTTAATGGTCTGAAAAAACTCGAGCACCACCACCACCACCACTGAGAT
CCGGCTGCTAA.

An especially preferred aspect relates to the endolysin where the stop codon was removed from the 3′ end of the gene to accommodate a 6×Histidine tag.

Polypeptide sequence of Endolysin with C-terminal Histidine tag (×6)

MRYIPAAHHSAGSNHPVNRVIHATCPDVGFPSASRKGRAVSTANYFASP
SSGGSAHYVCDIGETVQCLSEGTIGWHAPPNPHSLGIEICADGGSHASF
RVPGHAYTREQWLDPRVWPAVEKAAILCRRLCDKYNVPKRKLSAADLKA
GRRGVCGHVDVTDAWHQSDHDDPGPWFPWDRFMAVVNGHNESGELTVAD
VKALHDQIKQLSAQLAGSVNKLHHDVGWQVQNGDLGKRVDALSVWKNPV
TGKLWRTKDALWSVWYYVLECRSRIDRLESAVNGLKKLEHHHHHH

It is also possible that the nucleic acid molecules of the endolysin for optional inclusion in the composition of the invention comprise fragments, variants and fusions of the endolysin which are capable of specifically binding to and/or lysing cells of P. acnes. The unique ability of this endolysin is that it does not target any of the regular bacterial flora found on human skin namely S. aureus, S. epidermidis or E. coli, but is selectively found to target P. acnes. The phage derived endolyins are preferably included in 0.005 to 2.0%, more preferably 0.01 to 1% by weight of the composition.

The composition of the invention may be delivered on to the skin through a leave-on composition or a wash off composition, with the leave-on composition being more preferred. By a leave-on composition is meant a composition which is applied on to the desired surface of the skin and left thereon till the person washes a few hours and sometimes only a day later in the course of normal face or body wash. A leave-on composition generally comprises low amount of surfactant which is preferably less than 6%, more preferably less than 3% by weight of the composition. By a wash off composition is meant a composition that comprises high amount of surfactant preferably in the range of 6 to 80% and is generally applied on the desired surface of the skin after diluting the composition with water to generally wash off dirt, oil and other undesirable soil deposited on it.

The composition of the invention includes a topically acceptable carrier which comprises a non-ionic polymer. It may be delivered in the form of an anhydrous base, a gel, a lotion, a cream or an emulsion. The non-ionic polymer is preferably selected from any one of a hydroxypropyl methyl cellulose, hydroxyethyl cellulose, vinyl acetate/vinyl pyrrolidone copolymer, methocel, guargum, hydrophobically modified cellulose, hydrophobically modified polyurethane (e.g. Aculyn 44 or Aculyn 46) or hydrophobically modified polyether (e.g. Aculyn 60). The most preferred non-ionic polymer for inclusion in the composition of the present invention are hydroxypropyl methyl cellulose or hydroxyethyl cellulose.

The above two polymers have the structures as given below:

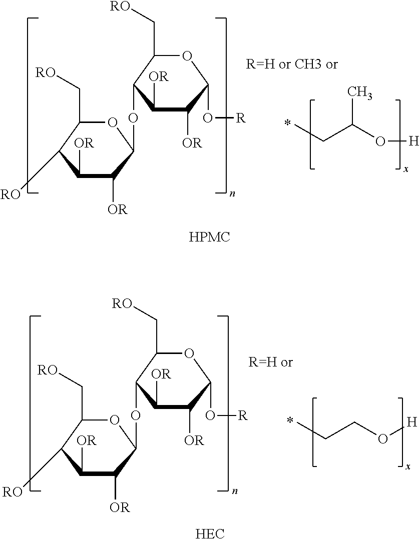

The non-ionic polymer is preferably included in 0.1 to 1.0% more preferably 0.2 to 0.6% by weight of the composition. It is especially preferred that polymers which have a net charge e.g. anionic or cationic polymers are included in very minimal amounts. By minimal amounts is meant that such ionic polymers are included in less than 0.1% preferably less than 0.01% and optimally absent from the composition. In other words, it is preferred that the composition is substantially free of anionic or cationic polymers.

It is especially preferred that the pH of the composition is between 5 and 9, more preferably in the range of 5.5 to 8.5, and most preferably in the range of 6 to 8. It has been observed that outside this range the effectiveness of the composition drops dramatically. pH is measured using the following protocol.

The pH measurement was carried out using a glass electrode-based pH-meter system of the composition without any dilution. The glass electrode was calibrated before the measurement with buffer standards. The glass electrode was dipped in 20 grams of the composition and allowed to equilibrate till a stable reading was obtained. This reading was recorded as the pH of the composition.

It is preferred that the composition of the invention comprises at the most 50 mM, more preferably at the most 30 mM, most preferably at the most 20 mM electrolyte. By an electrolyte is meant a compound that dissociates into ions in water. Examples of electrolyte are alkali metal salts like sodium sulfate, sodium chloride, sodium acetate, sodium citrate, potassium chloride, potassium sulfate, sodium carbonate and other mono or di or tri salts of alkaline earth metals. Electrolytes also include polyelectrolytes.

Water may preferably be present in 10 to 90% by weight of the composition depending on the format of the composition. In solid compositions water may be present in 10-30%, while in liquid or semi-solid composition, water may be present in 40 to 90%.

When the composition in accordance with the invention is a leave on composition, it preferably comprises one or more of fragrance, surfactant, organic sunscreen, inorganic sunscreen, emollient, humectant, extender pigment and preservative.

The carrier acts as diluent or dispersant for the ingredients of the compositions. The carrier may be aqueous-based, anhydrous or an emulsion, whereby a water-in-oil or oil-in-water emulsion is generally preferred. If the use of water is desired, water typically makes up the balance of the composition, which most preferably is from 40 to 80% by weight of the composition.

In addition to water, organic solvents may optionally be included as carrier to assist any other carrier in the compositions of the present invention. Examples include alkanols like ethyl and isopropyl alcohol.

Other suitable organic solvents include ester oils like isopropyl myristate, cetyl myristate, 2-octyldodecyl myristate, avocado oil, almond oil, olive oil and neopentylglycol dicaprate. Typically, such ester oils assist in emulsifying the compositions, and an effective amount is often used to yield a stable, and most preferably, water-in-oil emulsion.

Emollients may also be used, if desired, as a carrier. Alcohols like 1-hexadecanol (i.e. cetyl alcohol) are preferred. Other emollients include silicone oils and synthetic esters. Silicone oils suitable for use include cyclic or linear polydimethylsiloxanes containing from 3 to 9, preferably from 4 to 5 silicon atoms. Non-volatile silicone oils useful as emollients include polyalkyl siloxanes, polyalkylaryl siloxanes and polyether siloxane copolymers. The non-volatile polyalkyl siloxanes useful polydimethylsiloxanes. Silicone elastomers may also be used. The ester emollients that may optionally be used are:
(i) alkenyl or alkyl esters of fatty acids having 10 to 20 carbon atoms. Examples thereof include isoarachidyl neopentanoate, isononyl isonanonoate, oleyl myristate, oleyl stearate, and oleyl oleate;
(ii) ether-esters such as fatty acid esters of ethoxylated fatty alcohols;
(iii) polyhydric alcohol esters. Ethylene glycol mono and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty esters, ethoxylated glyceryl mono-stearate, 1,3-butylene glycol monostearate, 1,3-butylene glycol distearate, polyoxyethylene polyol fatty acid ester, sorbitan fatty acid esters, and polyoxyethylene sorbitan fatty acid esters are satisfactory polyhydric alcohol esters;
(iv) wax esters such as beeswax, spermaceti, stearyl stearate and arachidyl behenate; and,
(v) sterols esters, of which cholesterol fatty acid esters are examples.

Emollients, when present, typically make up from 0.1 to 50% by weight of the composition, including all ranges subsumed therein.

Fatty acids having from 10 to 30 carbon atoms may also be included as carriers. Examples of such fatty acids include pelargonic, lauric, myristic, palmitic, stearic, isostearic, oleic, linoleic, arachidic, behenic or erucic acid and mixtures thereof.

Humectants of the polyhydric alcohol type may also be employed in the compositions. The humectant often aids in increasing the effectiveness of the emollient, reduces scaling at the skin surface, stimulates removal of built-up scale and improves skin feel. Typical polyhydric alcohols include glycerol, polyalkylene glycols and more preferably alkylene polyols and their derivatives, including propylene glycol, dipropylene glycol, polypropylene glycol, polyethylene glycol and derivatives thereof, sorbitol, hydroxypropyl sorbitol, hexylene glycol, 1,3-butylene glycol, 1,2,6-hexanetriol, ethoxylated glycerol, propoxylated glycerol and mixtures thereof. For best results, the humectant is preferably propylene glycol or sodium hyaluronate. Other humectants which may be used include hydroxyethyl urea. The amount of humectant may be 0.2 to 25% by weight and preferably from 0.5 to 15% by weight of the composition.

Moisturization may be improved through use of petrolatum or paraffin.

Various other ingredients may also be used in compositions. Actives are defined as skin benefit agents other than emollients and other than ingredients that merely improve the physical characteristics of the composition. Although not limited to this category, general examples include extender pigments such as talcs and silicas, as well as alpha-hydroxy acids, beta-hydroxy acids and zinc salts.

Beta-hydroxy acids include salicylic acid. Zinc oxide and zinc pyrithione are examples of useful zinc salts.

Compositions, especially those containing water, need to be protected against harmful microorganisms. Anti-microbial compounds, such as triclosan, and preservatives may become necessary. Suitable preservatives include alkyl esters of p-hydroxybenzoic acid, hydantoin derivatives, propionate salts, and a variety of quaternary ammonium compounds. Particularly preferred preservatives are methyl paraben, propyl paraben, phenoxyethanol and benzyl alcohol. Preservatives are from 0.1 to 2% by weight of the composition.

The packaging could be a patch, bottle, tube, roll-ball applicator, squeeze container or lidded jar.

When the composition, in a less preferred embodiment of the invention, is formulated as a wash-off composition it preferably includes non-ionic surfactants. A suitable non-ionic surfactant are C8-C22, preferably C8-C16 fatty alcohol ethoxylates, comprising between 1 and 8 ethylene oxide groups. Suitable surfactant concentrations in liquid forms of cleaning application are generally more than 0.5 but less than 10%, preferably from 1 to 5% by weight of the composition. In solid compositions, the surfactant is preferably present in 5 to 40%, preferably from 10 to 30% by weight of the composition.

According to another aspect of the present invention there is provided a method of controlling or eradicating P. acnes from skin comprising the step of applying a composition of the first aspect on to a desired skin surface. The method is preferably non-therapeutic. The method is especially useful for reducing or eliminating acne on skin.

The invention will now be illustrated with the help of the following non-limiting examples.

### EXAMPLES

### Examples 1-4: Effect of Type of Polymer in the Formulation on Lytic Activity

Compositions as shown in Table 1 below were prepared:

TABLE 1

| | Example 1, | Example 2, | Example 3, | Example 4, |
|---|---|---|---|---|
| Ingredient | wt % | wt % | wt % | wt % |
| Propylene | 4.0 | 4.0 | 4.0 | 4.0 |
| glycol | | | | |
| Glycerol | 1.0 | 1.0 | 1.0 | 1.0 |
| EDTA | 0.04 | 0.04 | 0.04 | 0.04 |
| Polymer | HPMC | HEC | Xanthan | Aristoflex |
| | | | gum | AVC |
| Nature of | Nonionic | Nonionic | Anionic | Anionic |
| polymer | | | | |
| Polymer, | 1.0 | 1.0 | 1.0 | 1.0 |
| wt % | | | | |
| Water | To 100 | To 100 | To 100 | To 100 |

In the above Table,

HPMC is Hydroxy propyl methyl cellulose

HEC is Hydroxy ethyl cellulose

Aristoflex AVC is Ammonium AcryloyldimethyltaurateNinyl Pyrrolidone Copolymer

Preparation of the Endolysin:

The endolysin was expressed and purified from the clone using the standard molecular biology techniques. Briefly, Clone was propagated in 2-litre LB broth with ampicillin and once the OD has reached to 0.8, the culture was induced with 1 mM IPTG. The induction was done for 4 hours followed by harvesting the cells. The cell pellet was lysed using the lysis buffer (20 mM Tris buffer pH 8) by sonication (4×30 sec cycle, with 1 min intermittent cooling). Endolysin from the lysed cell supernatant was purified from the E. coli host proteins using Ni-NTA affinity chromatography as per the standard protocol using denaturing conditions. The purified protein was refolded using the step wise Urea gradient and final elution was done in HEPES buffer pH 7. Recombinant endolysin concentration post dialysis was measured by Bradford method. The lytic activity of purified lysin was evaluated using Turbidity reduction assay and Contact kill assay with bacterial cells.

Lytic Activity:

The bacterial cultures of C. acnes 6919 was washed twice with saline and resuspended in HEPES buffer (pH 7.2) and OD₆₂₀ₙₘ was adjusted to 0.6. The respective polymer-based gel formulation was weighed in sterile glass vial with magnetic stirrer inside. The endolysin was topped up in base formulation to get the final concentration as 100 μg/ml. 100 μl of OD adjusted culture was added to the formulation and the mixture was incubated at 37° C. for 1 hour with constant stirring. Respective controls like Base without active and lysin in buffer were kept. Post incubation, 1 ml of suspension was neutralized in D/E broth and serial 10-fold dilutions were prepared in 1× sterile saline. The plating was done using pour plate method and plates were incubated at 37° C. in anaerobic conditions for 5-7 days. The plates were counted, and colony counts were reported as Log CFU/ml. The relative log reductions were calculated with respect to control. Lytic activity (%) was calculated with reference to log reductions for Endolysin in HEPES buffer (pH 7.0) for the polymer-based gel formulations with endolysin top-up. The lytic activity (%) normalized to activity in HEPES buffer at pH 7.0 is summarized in the Table-2 below:

TABLE 2

| Example | Lytic activity (%) |
|---|---|
| 1 | 93.9 |
| 2 | 69.7 |
| 3 | 31.8 |
| 4 | 26.4 |

The data in the table above indicates that the P. acnes phage derived endolysin is more effective when formulated in a non-ionic polymer (Example 1,2) as compared to formulating in an ionic polymer (Examples 3, 4).

### Examples 5-11: Effect of pH

The bacterial cultures of C. acnes 6919 was washed twice with saline and resuspended in HEPES buffer (pH 7.2) and OD₆₂₀ₙₘ was adjusted to 0.6. Buffers covering pH range from 5 to 10 were prepared freshly. 20 mM Sodium acetate buffer for pH 5 & 6, 20 mM HEPES Buffer for pH 7, 20 mM Tris buffer for pH 8 & 9, 100 mM Sodium Carbonate-Bicarbonate buffer pH 10 & 11. The bacterial cultures were prepared in fresh buffer and OD was adjusted to 0.6 @ 620 nm. 100 μg/ml of lysin was added to bacterial cell suspension and absorbance was recorded every 15 minutes. The % lytic activity was calculated by comparing the starting OD for HEPES buffer pH 7 and the drop in OD at the end of experiment was considered as 100%. The drop in OD for rest of buffers were calculated as relative drop with respect to drop in pH 7 HEPES buffer and converted to % value. The % lytic activity was measured as mentioned above and summarized in the Table-3 below:

TABLE 3

| Example | 5 | 6 | 7 | 8 | 9 | 10 | 11 |
|---|---|---|---|---|---|---|---|
| PH | 4.0 | 5.0 | 6.0 | 7.0 | 8.0 | 9.0 | 10.0 |
| % lytic activity | 0 | 0 | 67.2 | 85.9 | 27.2 | 0.3 | 0 |

The data in the table 3 above indicates that the best efficacy is observed in the pH range of 6 to 8.

### Examples 12-16: Effect of Electrolyte Concentrations

The bacterial cultures of C. acnes 6919 was washed twice with saline and resuspended in HEPES buffer (pH 7.2) and OD₆₂₀ₙₘ was adjusted to 0.6. 20 mM HEPES Buffers (pH 7) containing sodium chloride range from 20 to 100 mM were prepared freshly. The bacterial cultures were prepared in fresh buffer with the increasing salt concentration from 20 to 100 mM and OD was adjusted to 0.6 @ 620 nm. The suspension in 20 mM HEPES buffer with lysin was used as a positive control. 100 μg/ml of lysin was added to bacterial cell suspension and absorbance was recorded every 15 minutes. The % lytic activity was calculated by comparing the starting OD for HEPES buffer pH 7 and the drop in OD at the end of experiment was considered as 100%. The drop in OD for rest of buffers were calculated as relative drop with respect to drop in pH 7 HEPES buffer and converted to % value.

The % lytic activity was measured as mentioned above and summarized in the Table-4 below:

TABLE 4

| Example | 12 | 13 | 14 | 15 | 16 |
|---|---|---|---|---|---|
| NaCl | 20 | 30 | 40 | 50 | 100 |
| concentration, mM | | | | | |
| % lytic activity | 84.3 | 32.3 | 19.3 | 14.3 | 0 |

The data in the table 4 above indicates that the efficacy starts to improve as one goes down in electrolyte concentration below 50 mM and is best at or below 20 mM.

1.-13. (canceled)

14. An anti-acne personal care composition comprising:
(i) P. acnes phage-derived endolysin comprising a nucleotide sequence of SEQ ID NO: 1; and
(ii) a topically acceptable carrier comprising a non-ionic polymer, wherein the composition comprises, at most, 50 mM of an electrolyte.

15. The composition of claim 14, wherein the non-ionic polymer is selected from any one of a hydroxypropyl methyl cellulose, hydroxyethyl cellulose, vinyl acetate/vinyl pyrrolidone copolymer, methocel, guar gum, hydrophobically modified cellulose, hydrophobically modified polyurethane, hydrophobically modified polyether or any combinations or mixtures thereof.

16. The composition of claim 14, wherein the non-ionic polymer is hydroxypropyl methyl cellulose or hydroxyethyl cellulose.

17. The composition of claim 14, wherein the composition is substantially free of an anionic polymer or cationic polymer.

18. The composition of claim 14, wherein the composition has a pH between 5 to 9.

19. The composition of claim 14, wherein the composition has a pH between 6 to 8.

20. The composition of claim 14, wherein the composition comprises, at most, 30 mM of the electrolyte.

21. The composition of claim 14, wherein the composition comprises, at most, 20 mM of the electrolyte.

22. A method of controlling or eradicating P. acnes from skin comprising the step of applying the composition of claim 14 to the desired skin surface.

23. A method for reducing or eliminating acne on skin comprising the step of applying the composition of claim 14 to the desired skin surface.


SEQUENCE LISTING (2 sequences)

SEQ ID NO: 1 - DNA, 893 bp, Artificial Sequence
  Nucleotide sequence of endolysin
atgcgttatattccggcagcacatcattcagcaggtagcaatcatccggttaatcgtgttgttattcatgcaacctgtccggatgttggttttccgagcgcaagccgtaaaggtcgtgcagttagcaccgcaaactattttgcaagcccgagcagcggtggtagcgcacattatgtttgtgatattggtgaaaccgttcagtgtctgagcgaaggcaccattggttggcatgcaccgcctaatccgcatagcctgggtattgaaatttgtgcagatggtggtagccatgcaagctttcgtgttccgggtcatgcatatacccgtgaacagtggctggatccgcgtgtttggcctgcagttgaaaaagcagcaattctgtgtcgtcgtctgtgcgataaatacaatgttccgaaacgtaaactgagcgcagcagatctgaaagcaggtcgtcgtggtgtttgtggtcatgttgatgttaccgatgcatggcatcagagcgatcatgatgatccgggtccgtggtttccgtgggatcgttttatggcagttgttaatggtcataacgaaagcggtgaactgaccgttgcagatgttaaagcactgcatgatcagattaaacagctgagcgcacagctggcaggtagcgttaataaactgcatcatgatgttggtgttgttcaggttcagaatggtgatctgggtaaacgtgttgatgcactgagctgggttaaaaatccggttaccggtaaactgtggcgtaccaaagatgcactgtggtcagtttggtattatgttctggaatgtcgtagccgtattgatcgtctggaaagcgcagttaatggtctgaaaaaactcgagcaccaccaccaccaccactgagatccggctgctaa

SEQ ID NO: 2 - protein, 292 aa, Artificial Sequence
  Polypeptide sequence of Endolysin with C-terminal Histidine tag (x6)
MRYIPAAHHSAGSNHPVNRVVIHATCPDVGFPSASRKGRAVSTANYFASPSSGGSAHYVCDIGETVQCLSEGTIGWHAPPNPHSLGIEICADGGSHASFRVPGHAYTREQWLDPRVWPAVEKAAILCRRLCDKYNVPKRKLSAADLKAGRRGVCGHVDVTDAWHQSDHDDPGPWFPWDRFMAVVNGHNESGELTVADVKALHDQIKQLSAQLAGSVNKLHHDVGVVQVQNGDLGKRVDALSWVKNPVTGKLWRTKDALWSVWYYVLECRSRIDRLESAVNGLKKLEHHHHHH